(19) United States
(12) Patent Application Publication
(10) Pub. No.: US 2023/0255561 A1
(43) Pub. Date: Aug. 17, 2023

(54) MINIMALLY-INVASIVE MONITORING PATCH

(71) Applicant: QuLab Medical Ltd., Herzliya (IL)

(72) Inventors: Idan TAMIR, Herzliya (IL); Sharon LEFLER, Herzliya (IL); David SCHREIBER, Herzliya (IL); Hila MASASA, Herzliya (IL); Berta BEN-SHACHAR, Herzliya (IL); Gil PERLBERG, Herzliya (IL)

(21) Appl. No.: 18/164,081

(22) Filed: Feb. 3, 2023

Related U.S. Application Data
(63) Continuation of application No. PCT/IB2021/000528, filed on Aug. 3, 2021.
(60) Provisional application No. 63/060,348, filed on Aug. 3, 2020.

(51) Int. Cl. A61B 5/00

(52) U.S. Cl. CPC A61B 5/685; A61B 5/6833; A61B 2562/046

(57) ABSTRACT

A wearable sensor patch including a generally cylindrical base having a bore, and a skin contact surface having an adhesive thereon; a piston-like part positioned within the bore; at least one microprobe positioned on the piston-like part; a retention spring; wherein the piston-like part is movable within the bore of the base between (1) a first position in which the at least one microprobe is positioned within the bore, and (2) a second position in which the at least one microprobe protrudes past the skin contact surface, and wherein the retention spring and the piston-like part are configured to cooperate such that the retention spring retains the piston-like part in either the first position or the second position

### CROSS-REFERENCE TO RELATED APPLICATION

This application is an international (PCT) patent application relating to and claiming the benefit of commonly-owned, co-pending U.S. Provisional Patent Application No. 63/060,348, filed on Aug. 3, 2020 and entitled “Minimally-Invasive Monitoring Patch,” the contents of which are incorporated herein by reference in their entirety.

### FIELD

The present disclosure relates to a wearable patch including one or more microprobes.

### BACKGROUND

Bio-analyte sensing, as well as drug delivery, using microprobes and microneedles (respectively) has the advantage of minimal invasiveness. Micro-sensing systems, such as sensors mounted on microneedles, microprobes or neural probes are commonly used for healthcare applications (among other). The minimal invasive approach has the dual advantage of inflicting less pain and being less prone to infections. In order to achieve reliable bio-analyte sampling and efficient drug delivery, the needles and microprobes must maintain a fixed depth and position in the skin. As microprobes are designed for shorter penetration depths, they become more susceptible to being ejected by the skin.

### SUMMARY OF THE INVENTION

This summary is a high-level overview of various aspects of the invention and introduces some of the concepts that are further detailed in the Detailed Description section below. This summary is not intended to identify key or essential features of the claimed subject matter, nor is it intended to be used in isolation to determine the scope of the claimed subject matter. The subject matter should be understood by reference to the appropriate portions of the entire specification, any or all drawings, and each claim.

In some embodiments of the present disclosure relate to an integrated biosensor wearable patch, including a linear microprobe array that is mounted on a substrate, power, electronics & communication, and an applicator by which the patch is mounted on the skin. The wearable patch may have an as-mounted low-profile, whilst the microprobes are inserted in the dermis or epidermis. The microprobes may be held at a fixed position, depth, and orientation, in the dermis or epidermis. The microprobes may be held by applying a force that can withstand un-intended ejection of the microprobes from the skin, due to skin and or muscle dynamics. Such skin-ejection opposing forces may be applied using a spring and or adhesive. The wearable patch may include a stopper mechanism to prevent the microprobe from being inserted deeper than intended into the skin. Such a stopper prevents the linear microprobe array from cutting into the skin due to skin movement, muscle motion and or unintended forces acting on the patch. The wearable patch may be designed to provide the linear microprobe array with a limited range of movement with respect to the patch shell, while being connected to the shell using elastic members that provide the linear microprobe array with a range of independent motion. The probes may be inserted at a sharp (<90 deg) angle to the skin. The probes may be inserted at an angle to the skin whilst the patch orientation is in sync with the body part and range of skin/muscle motion. Skin insertion requires a larger force than keeping the microprobe system in its initial skin location overtime. The patch therefore includes a two-spring mechanism. One spring for microprobe insertion and the other for keeping the microprobe in the skin for the entire patch wear duration. The microprobe tip or stem may include a bulge or barb, that will secure microprobe position in the skin over time. Such skin anchoring mechanism will maintain sensor skin penetration depth and position regardless of skin and muscle movement and prevent unintended ejection of the probes or a possible infection. The wearable patch shell may include an “adhesion force shaft”, which applies an axial force to the microprobe holder. A safety mechanism limiting microprobe skin penetration may be in place to prevent skin damage due to extensive microprobe substrate skin penetration. The insertion mechanism may be internal to the wearable patch shell. The applicator may be integrated within the wearable patch shell. The insertion mechanism may be external to the wearable patch shell. A small, simple, and cheap applicator may be used to mount the wearable patch onto the skin. The external insertion mechanism may be disposable.

Some-embodiments of the present disclosure also relate to another integrated biosensor wearable patch. The patch is comprised of a casing including a top shell, a patch base and an adhesive layer. The patch is also comprised of electronic components including a power source, an amplifier, communications devices and connectors. The patch is also comprised of a movable microprobe platform including a microprobe system, a microprobe system holder, a microprobe skin stopper, an adhesive layer, and electrical connectors. The patch is also comprised of a spring actuated microprobe array insertion mechanism, a latch that activates the spring actuated microprobe array insertion mechanism, and a safety mechanism that prevents unintended release of the spring actuated microprobe array insertion mechanism.

In some embodiments, upon release of the safety mechanism and upon activating the latch, the spring actuated microprobe array insertion mechanism is configured to move the microprobe platform towards the skin by a predetermined force.

In some embodiments, the patch includes at least one microprobe that is configured to be inserted into the skin.

In some embodiments, the patch is configured to insert at least one microprobe a predetermined depth into the skin.

In some embodiments, the microprobe skin stopper is configured to limit the travel of the movable platform.

In some embodiments, microprobe skin stopper is configured to limit the travel of the microprobe array.

In some embodiments, the movable platform is configured to be in contact with skin when at least one microprobe is inserted in the skin.

In some embodiments, the movable platform is configured to be in contact with skin when at least one microprobe is inserted at a predetermined depth in the skin.

Some embodiments of the present disclosure also relate to another integrated biosensor wearable patch. The patch is comprised of a casing including a top shell, a base and an adhesive layer. The patch is also comprised of electronic components including a power source, an amplifier, communications devices, and connectors. The patch is also comprised of a movable microprobe platform including a microprobe system, a microprobe system holder, a microprobe skin stopper, an adhesive layer, and electrical connectors. The patch is also comprised of a multiple-spring actuated microprobe array insertion mechanism, a spring motion limiter, a latch that activates the multiple-spring actuated microprobe array insertion mechanism, and a safety mechanism that prevents unintended release of the multiple-spring actuated microprobe array insertion mechanism.

In some embodiments, upon release of the safety mechanism and upon activating the latch, the multiple-spring actuated microprobe array insertion mechanism is configured to move the platform towards the skin with a predetermined force of the combined springs.

In some embodiments, the multiple-spring actuated microprobe array insertion mechanism includes two springs.

In some embodiments, the multiple-spring actuated microprobe array insertion mechanism, includes more than two springs.

In some embodiments, the multiple-spring actuated microprobe array insertion mechanism includes a first spring and a second spring, and the spring motion limiter is configured to limit the travel of the first spring, such that the first spring stops applying force on the movable microprobe platform, while the second spring continues to be in contact with the platform and applies a force of the second spring.

In some embodiments, the second spring is configured to apply an ejection-counterforce that resists forces that may move microprobes from their skin position or eject the microprobes from the skin.

In some embodiments, the first spring is configured to apply a force that is more than double of a force applied by the ejection-counterforce.

In some embodiments, the first spring is configured to apply a force that is more than triple of the force applied by the ejection-counterforce.

In some embodiments, the first spring is configured to apply a force that is more than quadruple of the force applied by the ejection-counterforce.

In some embodiments, the first spring is configured to apply a force that is more than quintuple of the force applied by the ejection-counterforce.

In some embodiments, the multiple springs are configured to apply a combined force that is more than 50 grams.

In some embodiments, the multiple springs are configured to apply a combined force that is more than 75 grams.

In some embodiments, the multiple springs are configured to apply a combined force that is more than 100 grams.

In some embodiments, the multiple springs are configured to apply a combined force that is more than 150 grams.

In some embodiments, the multiple springs are configured to apply a combined force that is more than 200 grams.

In some embodiments, the multiple springs are configured to apply a combined force that is more than 250 grams.

Some embodiments of the present disclosure also relate to another integrated biosensor wearable patch. The patch is comprised of a casing including a top shell, a base, an adhesive layer, and a leaf spring. The patch is also comprised of electronic components including a power source, communications devices, and connectors. The patch is also comprised of a movable microprobe platform including a microprobe system, a microprobe array holder, a skin stopper, and an adhesive layer. The patch is also comprised of a flexible connector connecting the movable microprobe platform and the base, a microprobe array insertion mechanism including a leaf spring connected to the top shell, and a push button latch positioned in the top shell above the leaf spring.

In some embodiments, the latch is configured to flip the leaf spring that forces the movable platform downwards when the push button is pressed.

In some embodiments, the flexible connector is connected to the movable microprobe platform above the patch base plane, such that it is at an angle to the plane of the base.

In some embodiments, the flexible connector is connected to the movable microprobe platform above the patch base plane, such that a force applied by the flexible connector has a vertical component that is directed towards the skin.

Some embodiments of the present disclosure also relate to a biosensor wearable patch system. The system is comprised of a disposable applicator including a shell, a push button latch, a slot, and a leaf spring. The system is also comprised of a casing including a top shell, a base and an adhesive layer. The system is also comprised of electronic components including a power source, an amplifier, communications devices, and connectors. The system is also comprised of a movable microprobe platform including a microprobe system, a microprobe system holder, a microprobe skin stopper, an adhesive layer, and electrical connectors. The system is also comprised of a flexible connector connecting the movable microprobe platform and the base and a microprobe array insertion mechanism including a leaf spring connected to the disposable applicator and push button latch positioned in the disposable applicator above the leaf spring.

In some embodiments, the latch is configured to flip the leaf spring that forces the movable platform downwards towards the skin when the push button is pressed.

In some embodiments, the flexible connector is connected to the movable microprobe platform above the base plane, such that it is at an angle to the plane of the base.

In some embodiments, the flexible connector is connected to the movable microprobe platform above the base plane, such that a force applied by the flexible connector has a vertical component that is directed towards the skin.

In some embodiments, the disposable applicator can be removed from the skin.

Some embodiments of the present disclosure are also related to another integrated biosensor wearable patch. The patch is comprised of a casing including a top shell, a base and an adhesive layer. The patch is also comprised of electronic components including a power source, communications devices and connectors. The patch is also comprised of a movable microprobe platform including a microprobe system, a microprobe array holder, a skin stopper, and an adhesive layer. The patch is also comprised of a flexible connector connecting the movable microprobe platform and the casing, a spring-actuated microprobe array insertion mechanism, a latch that is configured to activate the spring-actuated microprobe array insertion mechanism, and a safety mechanism that is configured to prevent unintended release of the spring-actuated microprobe array insertion mechanism.

In some embodiments, the flexible connector connects the movable microprobe platform and the top shell.

In some embodiments, the flexible connector connects the movable microprobe platform and the base.

In some embodiments, the flexible connector is configured to limited changes to the position and orientation of the movable microprobe platform with respect to its casing.

In some embodiments, the flexible connector is configured to enable one or a combination of the following limited position and orientation movements of the movable microprobe platform: linear movements in the radial, lateral or vertical directions, rotations such as a yaw, pitch, and roll.

In some embodiments, the adhesive layer of the movable microprobe platform connects the movable microprobe platform to the skin, such that the position or orientation of the movable microprobe platform can conform to local changes in skin orientation, irrespective of the base position and or orientation.

In some embodiments, the flexible connector includes a spring.

In some embodiments, at least one side of the flexible connector is connected to an anchor.

In some embodiments, the flexible connector direction is from the center to the circumference in a radial fashion.

In some embodiments, the flexible connector extends from the center of the patch to the circumference in an off radial direction.

In some embodiments, the flexible connector is located on the same plane as the base.

In some embodiments, the flexible connector is configured to apply a force on the same plane as the base.

In some embodiments, the flexible connector is connected to the movable microprobe platform above the base plane, such that it is at an angle to the plane of the base.

In some embodiments, the flexible connector is connected to the movable microprobe platform above the base plane, such that a force applied by the flexible connector has a vertical vector component that is directed towards the skin.

Some embodiments of the present disclosure are also related to another biosensor wearable patch system. The system is comprised of a disposable applicator including a disposable applicator top shell with walls, an adhesive layer, an inserter, a safety mechanism, an inserter spring, and a slot. The system is also comprised of a patch. The patch includes casing including a base and an adhesive layer. The patch also includes electronic components including a power source, an amplifier, communications devices and connectors. The patch also includes a microprobe platform including a microprobe system including a microprobe system holder, a microprobe skin stopper, an adhesive layer, and electrical connectors.

In some embodiments, the system is configured such that while the safety mechanism is in place, the microprobes are configured to be held within a volume defined by the disposable applicator top shell and disposable applicator walls.

In some embodiments, the system is configured such that releasing the safety mechanism enables the spring-actuated inserter to move the patch towards the skin.

In some embodiments, the system is also comprised of a flexible connector connecting the movable microprobe platform and the casing.

In some embodiments, the system is also comprised of a flexible connector connecting the movable microprobe platform and the base.

In some embodiments, the flexible connector is configured to enable changes to one or a combination of the following limited position and orientation of the movable microprobe platform: linear movements in the radial, lateral or vertical direction, rotations such as a yaw, pitch, and roll.

Some embodiments of the present disclosure also relate to another integrated biosensor wearable patch system. The system is comprised of an inserter system including an inserter system top shell, a rotation axis, a spacer, and a spring. The system is also comprised of a patch. The patch includes a casing including a base, an adhesive layer, and a cavity. The patch also includes electronic components including a power source, communications devices, and connectors. The patch also includes a movable microprobe platform including a microprobe system, a microprobe array holder, a skin stopper, an adhesive layer, and a friction plane, wherein the spring connects the inserter system and the base, wherein an opening in the surface of the cavity guides movement of the movable microprobe platform, and wherein the inserter system is configured to contact the movable microprobe platform on the friction plane.

In some embodiments, the patch, with the spacer in place, as measured in air, has a thickness that is larger than the height of the mounted integrated biosensor wearable patch above the skin.

In some embodiments, the patch, with the spacer in place, as measured in air, has a thickness that is larger than the height of the patch above the skin by more than the microprobe length, or Lmax.

In some embodiments, the patch has a thickness that is larger than the thickness of the base, by less than the spacer's thickness.

In some embodiments, when the spacer is removed, the inserter system is configured to move.

In some embodiments, when the spacer is removed, the inserter system is configured to rotate in the direction of an individual's skin.

In some embodiments, when the spacer is removed, a distal end of the inserter system is configured to move in the direction of an individual's skin.

In some embodiments, the top shell of the inserter system that is aligned with microbes and has a cross section that is one of U shaped, L shaped, flat, or rounded.

In some embodiments, the cross section of the inserter system top shell that is in line with the microprobes is L shaped, flat, rounded.

In some embodiments, a flexible connector connects the movable microprobe platform with the casing.

In some embodiments, a flexible connector connects the movable microprobe platform with the base.

In some embodiments, the flexible connector is configured to enable changes to one or a combination of the following limited positions and orientations of the movable microprobe platform: linear movements in the radial, lateral or vertical direction, rotations such as a yaw, pitch, and roll.

Some embodiments of the present disclosure are also related to another integrated biosensor wearable patch. The patch is comprised of a casing including a top shell, a base, and an adhesive layer. The patch is also comprised of electronic components including a power source, an amplifier, communications devices, and connectors. The patch is also comprised of a movable microprobe platform including a microprobe system, a microprobe system holder, a microprobe skin stopper, an adhesive layer, and electrical connectors. The patch is also comprised of an adhesive pressure shaft, wherein the adhesive is in contact with the movable microprobe platform and with the top shell of the casing. The patch is also comprised of a spring actuated microprobe array insertion mechanism, a latch that is configured to activate the spring actuated microprobe array insertion mechanism, and a safety mechanism that is configured to prevent unintended release of the spring actuated microprobe array insertion mechanism.

In some embodiments, the adhesive pressure shaft is configured enable a downwards force to be applied to the movable microprobe platform.

In some embodiments, the adhesive pressure shaft is configured to enable a downwards force to be applied to a component of the movable microprobe platform.

In some embodiments, the adhesive pressure shaft is configured to enable a downwards force to be applied to the movable microprobe platform independently of the top shell of the casing.

In some embodiments, the adhesive pressure shaft is configured to enable a downwards force to be applied to the movable microprobe platform while applying a downwards force to the top shell of the casing.

In some embodiments, the adhesive pressure shaft is configured to move independently of top shell of the casing.

In some embodiments, a top aspect of the adhesive pressure shaft protrudes above the top shell of the casing.

In some embodiments, a top aspect of the adhesive pressure shaft is connected to the top shell of the casing.

In some embodiments, the top shell of the casing includes a visible mark that identifies the location of the adhesive pressure shaft virtual axis.

Some embodiments of the present disclosure are also related to another integrated biosensor wearable patch. The patch is comprised of a casing including a top shell, a base and adhesive layer. The patch is also comprised of electronic components including a power source, an amplifier, communications devices, and connectors. The patch is also comprised of a movable microprobe platform, a microprobe system, a microprobe system holder, a microprobe skin stopper, an adhesive layer, and electrical connectors. The patch is also comprised of an adhesive pressure shaft, wherein the adhesive pressure shaft is in contact with the movable microprobe platform and with the top shell of the casing. The patch is also comprised of a spring actuated microprobe array insertion mechanism, a latch that activates the spring actuated microprobe array insertion mechanism, and a safety mechanism that prevents unintended release of the spring actuated microprobe array insertion mechanism.

In some embodiments, the adhesive pressure shaft is configured to a downwards force to be applied to the movable microprobe platform.

In some embodiments, the adhesive pressure shaft is configured to enable a downwards force to be applied to an element that is part of the movable microprobe platform.

In some embodiments, the adhesive pressure shaft is configured to enable a downwards force to be applied to the movable microprobe platform independently of the top shell of the casing.

In some embodiments, the adhesive pressure shaft is configured to enable a downwards force to be applied to the movable microprobe platform while applying a downwards force to the top shell of the casing.

In some embodiments, the adhesive pressure shaft is configured to move independently of the top shell of the casing.

In some embodiments, a top aspect of the adhesive pressure shaft protrudes above the top shell of the casing.

In some embodiments, a top aspect of the adhesive pressure shaft is connected to the top shell of the casing.

In some embodiments, the top shell of the casing includes a visible mark that identifies the location of the adhesive pressure shaft virtual axis.

In some embodiments, a wearable sensor patch includes a generally cylindrical base having a bore, and a skin contact surface having an adhesive thereon; a piston-like part positioned within the bore; at least one microprobe positioned on the piston-like part; a retention spring; wherein the piston-like part is movable within the bore of the base between (1) a first position in which the at least one microprobe is positioned within the bore, and (2) a second position in which the at least one microprobe protrudes past the skin contact surface, and wherein the retention spring and the piston-like part are configured to cooperate such that the retention spring retains the piston-like part in either the first position or the second position.

In some embodiments, the retention spring is configured to allow the piston-like part to move from the first position to the second position upon application of a sufficient release force to the piston-like part. In some embodiments, the sufficient release force is at least 0.1 kilogram.

In some embodiments, the at least one microprobe is positioned within a microprobe housing. In some embodiments, the microprobe housing includes a flex connector configured to connect the at least one microprobe to an external processing system. In some embodiments, the microprobe housing includes a metal support supporting the at least one microprobe. In some embodiments, the microprobe housing includes a housing first part and a housing second part encasing at least part of the at least one microprobe.

In some embodiments, the spring is a U-shaped spring.

In some embodiments, a sensor system includes the wearable sensor patch and an applicator. In some embodiments, the applicator includes an actuator configured to be actuated by a user; a plunger configured to contact the piston-like part of the wearable sensor patch when the applicator is assembled to the wearable sensor patch; and a spring configured to apply a force to the plunger when the actuator is actuated. In some embodiments, the spring is pre-loaded. In some embodiments, the spring is configured to be loaded upon application of the wearable sensor patch to skin of a user.

In some embodiments, the at least one microprobe includes a plurality of microprobes. In some embodiments, the plurality of microprobes are staggered so as to include a lead microprobe that contacts the skin before an additional microprobe. In some embodiments, the lead microprobe is positioned at a center of the plurality of microprobes. In some embodiments, the lead microprobe is positioned at an end of the plurality of microprobes.

In some embodiments, a wearable sensor patch includes a casing; a movable microprobe platform movably positioned within the casing, wherein the movable microprobe platform includes a microprobe system; and a multiple-spring microprobe array insertion mechanism including a plurality of springs, and a latch operable to activate the multiple-spring microprobe array insertion mechanism, wherein, when the latch is operated so as to activate the multiple-spring microprobe array insertion mechanism, the plurality of springs acts on the movable microprobe platform with a combined force of the plurality of springs so as to deploy the movable microprobe platform.

In some embodiments, the multi-spring microprobe array insertion mechanism includes a spring motion limiter, wherein the multiple-spring microprobe array insertion mechanism includes a first spring and a second spring, and wherein the spring motion limiter is configured to limit a travel of the first spring such that the first spring stops applying force to the movable microprobe platform when the first spring contacts the spring motion limiter while the second spring continues to be in contact with the movable microprobe platform and to apply a force of the second spring to the movable microprobe platform. In some embodiments, the second spring is configured to apply an ejection-counterforce sufficient to resists force that may move microprobes of the microprobe system from a skin position or eject the microprobes from skin. In some embodiments, the first spring is configured to apply a force that is more than two times the ejection-counterforce.

In some embodiments, a combined force applied by the plurality of springs is at least 50 grams.

In some embodiments, the plurality of springs includes a plurality of torsion springs. In some embodiments, the plurality of torsion springs have a same spring axis.

In some embodiments, the wearable sensor patch also includes a safety mechanism configured to prevent unintended release of the multiple-spring microprobe array insertion mechanism.

### BRIEF DESCRIPTION OF THE DRAWINGS

Some embodiments of the present disclosure are herein described, by way of example only, with reference to the accompanying drawings. With specific reference now to the drawings in detail, particulars shown are by way of example and for purposes of illustrative discussion of some embodiments of the invention. In this regard, the description taken with the drawings makes apparent to those skilled in the art how embodiments of the invention may be practiced.

FIG. 1 is a cross section view of a patch including a microprobe according to an exemplary embodiment of the present disclosure.

FIG. 2 is a cross section view of a microprobe according to an exemplary embodiment of the present disclosure.

FIG. 3 is a graph depicting insertion forces according to an exemplary embodiment of the present disclosure.

FIG. 4 is a cross section view of the internal portion of a patch according to an exemplary embodiment of the present disclosure.

FIG. 5 is a cross section view of a multi-spring insertion mechanism according to an exemplary embodiment of the present disclosure.

FIGS. 6A and 6B are cross-section views of pre-mount patch configurations according to exemplary embodiments of the present disclosure.

FIG. 7 is a cross-section view of a patch with a “cave-in” cover according to an exemplary embodiment of the present disclosure.

FIGS. 8A and 8B are cross section views of microprobe array designs according to exemplary embodiments of the present disclosure.

FIG. 9A is a top perspective view of an embodiment of a patch according to an exemplary embodiment of the present disclosure.

FIG. 9B is a bottom perspective view of the patch shown in FIG. 9A, the patch being shown in an undeployed position.

FIG. 9C is a bottom perspective view of the patch shown in FIG. 9A, the patch being positioned in a deployed position.

FIG. 10A is an exploded view of an embodiment of a microprobe holder according to an exemplary embodiment of the present disclosure.

FIG. 10B is a partially assembled view of the microprobe holder shown in FIG. 10A.

FIG. 10C is an assembled view of the microprobe holder shown in FIG. 10A.

FIG. 10D is a cross-section view of the microprobe holder shown in FIG. 10A.

FIG. 11A is a cross-section view of the patch shown in FIG. 9A assembled to an applicator according to an exemplary embodiment of the present disclosure, the patch and applicator being positioned in an undeployed position.

FIG. 11B is a cross-section view of the patch and applicator shown in FIG. 11A, the patch and applicator being positioned in a deployed position.

### DETAILED DESCRIPTION

The following description of the preferred embodiment(s) is merely exemplary in nature and is in no way intended to limit the invention, its application, or uses. As used throughout, ranges are used as shorthand for describing each and every value that is within the range. Any value within the range can be selected as the terminus of the range. In addition, all references cited herein are hereby incorporated by referenced in their entireties. In the event of a conflict between a definition in the present disclosure and that of a cited reference, the present disclosure prevails.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention.

Relative terms such as “lower,” “upper,” “horizontal,” “vertical,” “above,” “below,” “up,” “down,” “left,” “right,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such.

Terms such as “attached,” “affixed,” “connected,” “coupled,” “interconnected,” “mounted” and similar refer to a relationship wherein structures are secured or attached to one another either directly or indirectly through intervening structures, as well as both movable or rigid attachments or relationships, unless expressly described otherwise.

As used in the specification and the claims, the singular form of “a”, “an”, and “the” include plural referents unless the context clearly dictates otherwise.

Spatial or directional terms, such as “left”, “right”, “inner”, “outer”, “above”, “below”, and the like, are not to be considered as limiting as the invention can assume various alternative orientations.

All numbers used in the specification and claims are to be understood as being modified in all instances by the term “about”. The term “about” means a range of plus or minus ten percent of the stated value.

Unless otherwise indicated, all ranges or ratios disclosed herein are to be understood to encompass any and all subranges or subratios subsumed therein. For example, a stated range or ratio of “1 to 10” should be considered to include any and all subranges between (and inclusive of) the minimum value of 1 and the maximum value of 10; that is, all subranges or subratios beginning with a minimum value of 1 or more and ending with a maximum value of 10 or less, such as but not limited to, 1 to 6.1, 3.5 to 7.8, and 5.5 to 10.

The terms “first”, “second”, and the like are not intended to refer to any particular order or chronology, but instead refer to different conditions, properties, or elements.

All documents referred to herein are “incorporated by reference” in their entirety.

The term “at least” means “greater than or equal to”. The term “not greater than” means “less than or equal to”.

As used herein, the term “microprobe” is interchangeable with the terms “microneedle” and “neural probe”.

As used herein, the term “distal end” is defined as a distal-most point or line of a unit. For example, the distal end of a tip of a microprobe is the part of the microprobe that contacts the skin first.

As discussed herein, some embodiments of the present disclosure include an on-body wearable patch device that is designed to secure the in-skin position and penetration depth of an array of microprobes over the entire patch wear time. This patch is typically mounted on the skin for a period of several days or weeks. The wearable patch device is designed to exert a skin penetration force to counter forces resulting from skin motion as well as the natural tendency of skin to expel foreign objects.

In some embodiments, the patch may include a microprobe-based sensor array, electronics for signal capture and processing, and an insertion mechanism for the microprobe array so that the patch may transmit data collected by the sensors to a receiver on an external device such as a mobile phone, tablet, or smart watch. In some embodiments, the data may be further transmitted to a computation facility for further analysis and storage. For example, if the patch is being used for a medical purpose, the data can be forwarded to the patient's physician or health provider.

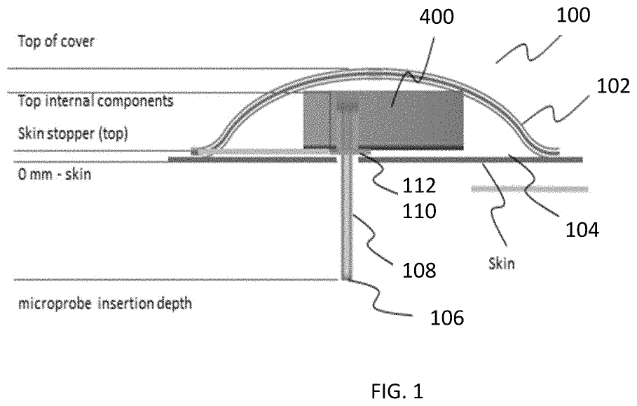

FIG. 1 depicts a cross section of an exemplary patch 100 mounted to skin with a microprobe inserted into the skin. The patch may be shaped so that it may be adhered to the skin and may prevent the worn patch from latching to clothes and/or bumping into objects. For example, as depicted in FIG. 1, in some embodiments, the patch 100 may be rounded.

FIG. 1 further depicts that the patch 100 may include a casing, which may include a top shell 102 and a base 104. In some embodiments, such as the embodiment depicted in FIG. 1, the patch may also include and a microprobe assembly 112. The microprobe assembly may include a microprobe 108 having a microprobe tip 106 and a skin stopper 110.

In some embodiments, the patch 100 may be equipped with microprobes that sense variations in the presence or concentrations of different bio-analytes (including, but not limited to, ions, metabolites, pH) in the dermal or subdermal tissue.

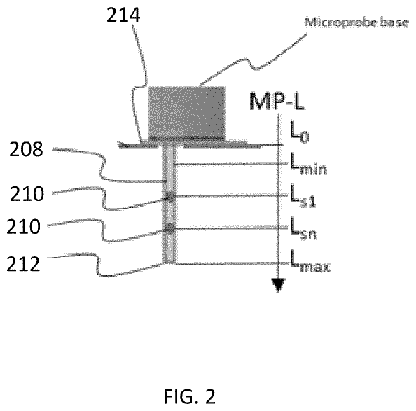

FIG. 2 depicts a microprobe 208 that is configured to measure bio-analyte concentrations in human skin in a minimally invasive manner. The microprobe 208 may include one or more sensors 210 that may be configured to measure the bio-analyte concentrations. The one or more sensors 210 may be positioned at any desired location along the microprobe 208. For example, in some embodiments, the sensors 210 on one microprobe may be in a different location that the sensors 210 on another microprobe, which may enable the microprobes to obtain a variety of measurements. The sensors 210 may further be positioned to obtain a desired signal quality, as well as sensing signal redundancy that may result from miss-location of one or more of the sensors, e.g., due to sensor localization in the epidermal tissue. The microprobe tip 212 may further be positioned at any desirable depth within the skin to measure the bio-analyte concentrations.

In some embodiments, the microprobe unit may include electronics (not shown) that enable communication with external devices, including, for example, computers and mobile devices. The electronics may include several components, including a power source, an amplifier, communications devices, and connectors.

The microprobe 208 depicted in FIG. 2 is inserted into an individual's skin. As shown in FIG. 2, in some embodiments, the microprobe 208 may include a microprobe skin stopper 214, which may directly or indirectly contact the outer skin surface to minimize and/or avoid tissue damage.

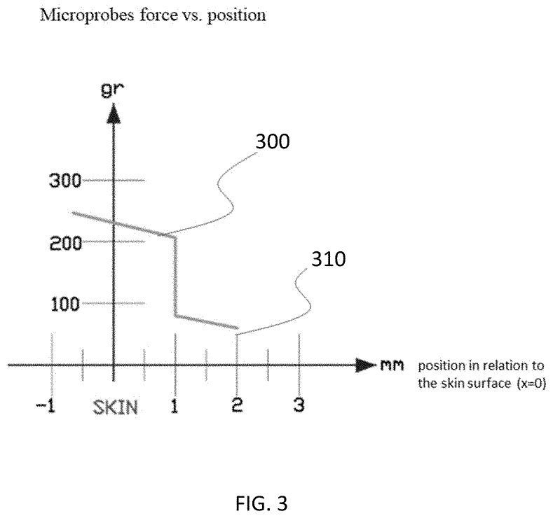

FIG. 3 depicts a graph that shows two skin insertion phases of inserting a microprobe into an individual's skin 300, 310. The first phase 300 employs a relatively fast-acting, high force mechanism for skin penetration. The second skin insertion phase 310 is a longer-acting, lower force mechanism designed to apply an additional retention/residual force to the microprobe over the course of days and/or weeks.

In the first insertion phase 300, following the triggering of a microprobe insertion mechanism, several hundred grams of force may be applied to the microprobe to allow efficient and rapid microprobe penetration of the skin. Then, in the second insertion phase 310, residual force is applied and maintained as long as the patch is being worn. The residual force enables the microprobe to maintain its position within the skin even under relative motion of the patch base with respect to the skin surface.

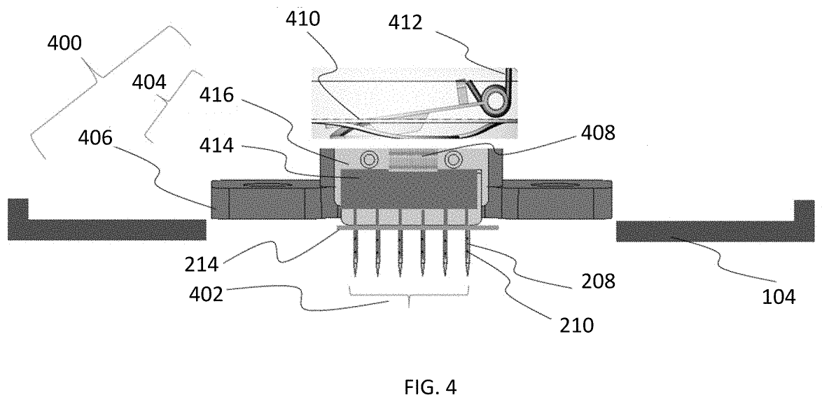

FIG. 4 depicts an exemplary movable microprobe platform 400. The platform 400 may be mechanically connected components that apply force to the insertion force (discussed above with respect to FIG. 3) to the microprobes 208. As depicted in FIG. 1, the platform 400 may be sized, shaped and configured to be placed within the patch 100, between the top shell 102 and the base 104. The platform may be configured to move as one single component in the direction of the applied insertion force. The non-limiting components of an exemplary platform are discussed in turn below.

The platform 400 may include a microprobe array 402 having one or more microprobes 208 attached there too. Each microprobe 208 in the microprobe array 402 may include one or more sensors 210. A distal end of each microprobe includes the microprobe tip which may assist in inserting the microprobe and corresponding one or more sensors into the skin. FIG. 4 depicts the microprobes 208 and sensors 210 of FIG. 2. However, the microprobes may be any suitable microprobe known to a person of ordinary skill in the art. For example, some embodiments of microprobes and microprobe arrays are described in U.S. provisional patent application 62/962,677, the entirety of which is incorporated herein by reference.

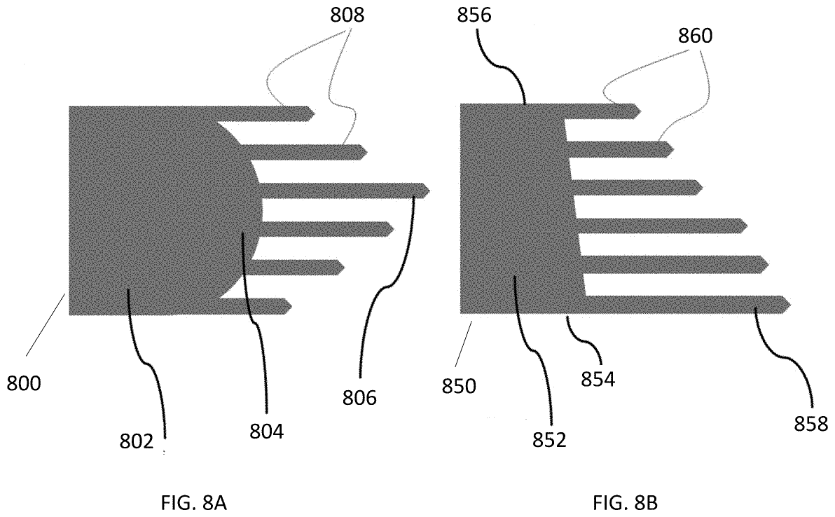

In addition, in some embodiments, the microprobe array 402 may be designed so that it can reduce microprobe array skin insertion pain and the force required for microprobe skin insertion. For example, in an exemplary embodiment depicted in FIG. 8A, the microprobe array 402 may be a staggered microprobe array 800. In some embodiments, the staggered microprobe array 800 includes a base 802 having a curved end 804 so as to include a lead microprobe 806 that contacts the skin first, followed by the additional array microprobes 808 that contact the skin thereafter.

Moreover, in some embodiments, such as the exemplary embodiment depicted in FIG. 8B, the microprobe array 402 may be a staggered microprobe array 850 that is designed so that the microprobes are configured to maintain their skin position and penetration depth. Such designs may include features that may reduce the possibility of microprobe ejection by the skin or another tissue. In some embodiments, the staggered microprobe array 850 includes a base 852 having an angled end including a longer side 854 and a shorter side 856, wherein the longer side 854 defines a lead microprobe 858 and the transition from the longer side 854 to the shorter side 856 defines trailing microprobes 860.

In some embodiments, the microprobes 208 may be are surface-coated by a conformal coating, which may function to anchor the microprobes 208 in the skin. In some embodiments, the microprobes may be spear shaped and include a barbed tip, allowing the microprobe to be easily inserted into the skin, while ejecting the microprobe by the skin will require additional forces to overcome the barbed-shaped microprobe tip.

As depicted in FIG. 4, in some embodiments, the platform 400 may further include a microprobe system 404, which may include a microprobe array substrate 414 and a microprobe array support 416. In some embodiments, the microprobe system 404 may be a structure having a flat component whose width and height may be larger than its thickness. As depicted in FIG. 4, in some embodiments, the microprobe system 404 may be positioned perpendicular to the skin. In other embodiments, the microprobe system 404 may be positioned at an angle smaller than 90 degrees versus the skin's plane, such that the microprobes can be inserted into the skin at a sharp angle.

The microprobe array substrate 414 may be a component to which the proximal end of each of the one or more microprobes is attached. The substrate may be any shape, size, or configuration so long as it is configured to connect to the proximal end of each microprobe.

The microprobe array support 416, which may be sized, shaped and/or configured to support the microprobe array 402 and/or the microprobe array substrate 414. The microprobe array support may further be configured to provide mechanical support and protection to the microprobe array 402 and/or the microprobe array substrate 414.

In some embodiments, the platform 400 may further include a skin stopper 214, which may be positioned between the microprobe array 402 and the microprobe array substrate 414 and/or microprobe array support 416 and/or microprobe array connector 408. Once the microprobes 208 are inserted into the skin, the skin stopper may be positioned in direct or indirect contact with the skin. The skin stopper 214 may be configured to limit the insertion depth of the microprobes 208 into the skin surface, while allowing changes to the temporal position of the skin surface with respect to the patch. For example, in some embodiments, the patch may allow the skin stopper 214 to be in direct contact with the skin (by a force or an adhesive), which may result in a fixed microprobe position on the skin's surface over the entire period the patch is being worn.

In some embodiments, the skin stopper 214 may include an adhesive on the surface which is configured to contact the skin. In some embodiments, the skin facing surface area of the skin stopper 214 may be larger than the horizontal cross section of the microprobe system 404.

In some embodiments, the skin stopper 214 may also function to protect the skin from tissue damage (e.g., cuts and bruises) resulting from lateral or rotational movements of the microprobe system 404. For example, in the absence of a skin stopper component, an edge of the microprobe array substrate 414 or the microprobe array support 416 may cut into the skin. In some embodiments, the skin stopper 214 may be configured to prevent the microprobe array substrate 414 or the microprobe array support 416 from reaching the skin even if excessive pressure is applied during microprobe insertion and during patch wearing, for example, to keep the sensors 210 on the microprobes 208 at a fixed position within the skin.

In some embodiments, the platform 400 may also include a microprobe array connector 408, which may be an electrical connector that may be configured to connect the microprobe sensors 210 to electronic components within the patch 100. In some embodiments, the microprobe array connector 408 may also provide electrical connectivity to other patch elements. In some embodiments, the microprobe array connector 408 may also provide mechanical connectivity, securing or connecting the microprobe array 402 to other patch components. In some embodiments, as depicted in FIG. 4, the microprobe array connector 408 may be mounted to the microprobe array support 416.

FIG. 4 further depicts that in some embodiments, the platform 400 includes a microprobe system holder 406. The microprobe array substrate 414 and the microprobe array support 416 may be mounted on or within the microprobe array holder 406. In some embodiments, the microprobe the microprobe array holder 406 constitutes the “moving part” of the patch 100. For example, using the insertion mechanism 410 (discussed below), the microprobe array holder 406 may move the microprobe system 404 towards the skin.

FIG. 4 also depicts that the platform 400 may include an insertion mechanism 410 for providing force to insert the microprobes 208 into the skin. In some embodiments, the insertion mechanism 410 may be connected on one side to the top shell 102 of the patch 100 and may be connected on the other side to the microprobe system holder 406. The connection of the insertion mechanism 410 to the top shell 102 may be direct or indirect, such as via other patch components. The connection of the insertion mechanism 410 to the top shell 102 may be a fixed connection, a pivot connection, or a contact/temporary connection.

In some embodiments, the insertion mechanism 410 may include an insertion force element 412. The insertion force element 412 may be any element configured to provide a force to the insertion mechanism 406. In some embodiments, for example, the insertion force element 412 may be a spring.

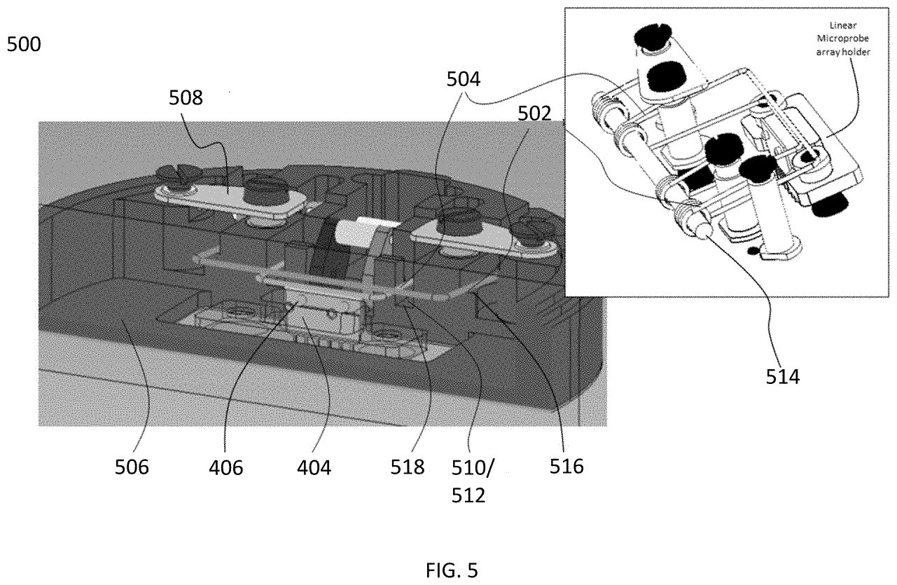

FIG. 5 depict an exemplary embodiment of an insertion mechanism having multiple springs. The multi-spring system 500 may be designed to exert differential forces at the two phases of patch activation (FIG. 3—insertion force 300 and residual force 310). As previously discussed, the first phase of microprobe insertion delivers a relatively large force allowing efficient microprobe insertion into the skin, and the second phase of microprobe insertion delivers reduced force to keep the microprobes in place throughout the entire period of patch wear, which may be between days and weeks.

In the exemplary embodiment depicted in FIG. 5, the multi-spring system 500 includes a first spring 502 and a second spring 504. The first spring 502 may exert the same, smaller, or larger force than the second spring 504. The two springs 502, 504 may be directly or indirectly fixed to the base 506 of the patch or using, for example, a counter plate 508 or any other holding or fixation elements.

The two springs 502, 504 may be positioned such that each has the same or separate motion slot. In the embodiment shown in FIG. 5, the first spring 502 and the second spring 504 have respective motion slots 510, 512 that are the same motion slot. The two springs 502, 504 may share the same spring axis bar or have different spring axis bars. In the embodiment shown in FIG. 5, the springs 502, 504 share the same spring axis bar 514. The two springs 502, 504 may further be designed such that movement of the microprobe system 404 driven by each of the springs 502, 504 encounters a motion limiter at a different location/phase of the entire linear microprobe array movement range. In the embodiment shown in FIG. 5, the first spring 502 has a motion limiter 516 and the second spring 504 has a motion limiter 518.

In the spring-loaded position, prior to microprobe array release, the two springs 502, 504 may press simultaneously against the linear microprobe system holder 406, applying their combined forces to this holder 406.

Following the release of the spring-loaded mechanism, the microprobe array 402 may be pushed into the skin through an opening in the patch base 104 to a predetermined penetration depth. The predetermined penetration depth may be less than 0.1 mm, less than 0.5 mm, less than 1.0 mm, less than 2.0 mm, less than 3.0 mm, or less than 4.5 mm.

Once the predetermined skin penetration depth has been achieved, the first spring arrives at the first motion limiter where it stops applying force to the microprobe system holder. At this point, the second spring continues to apply force to the microprobe system holder, thereby continuously pushing the microprobes into the skin throughout patch use duration.

In some embodiments, the second spring may also serve to compensate for external forces applied by the skin and/or patch, e.g., in the event of some skin displacement from the patch base. For example, the second spring may have a second spring motion limiter which may act as a failsafe mechanism to ensure that the skin will not endure excessive pressure from the microprobe holder that may lead to skin injury. In some embodiments, the motion limiter may also limit the movement of the microprobe array 402 out of the base 104 in case of unintended triggering of the mechanism.

As shown in FIG. 1 and FIG. 4, the patch may include a base 104. The base 104 may be sized, shaped and configured to support other components and subsystems of the patch 100. The base 104 may include a top side and a bottom side. The top side of the base 104 may be configured to be in direct or indirect contact with the shell 102 and/or with a wall of the shell 102.

The bottom side of the base may be configured to be positioned adjacent to the skin. In some embodiments, the bottom side of the base 104 includes an adhesive layer. The adhesive layer may be used to fix the base 104 to the skin. The adhesive layer may be uniform or have sections with different adhesive properties.

The base 104 of the patch may further be configured to provide a counter force to the force applied by the insertion mechanism 410, such that it may prevent the patch 100 from moving during microprobe insertion.

Further some embodiments of the patch of the present disclosure are discussed below with respect to an exemplary Continuous Glucose Monitoring (CGM) patch, which may be mounted on the skin for the duration of days and weeks. However, it should be appreciated that the patch may be any bio-analytics patch known to those skilled in the art. The CGM patch may include the same components as the patch depicted in FIGS. 1-4 and may further include additional components discussed below.

When an individual is wearing a patch, such as a CGM patch, the properties of skin in contact with the adhesive area on the base 104 may change. For example, skin maceration, blistering and other skin-related issues resulting from long term contact with adhesives may affect the properties of the skin under the patch. Changes in the skin properties may result in a relative motion between internal and external patch sections. Such relative motion may include lateral displacement as well as torsional or rotational movement of the internal section with respect to the external one.

Although these movements may be relatively small, they may lead to forces acting to eject the microprobes 208 from the skin. For example, a portion of a microprobe 208 may be ejected, thereby changing the sensor 210 position in the skin. A change in sensor 210 position in the skin might change and negatively affect signal integrity.

In addition, throughout the use of the patch, some change in skin dynamics might affect the tight interface between the patch base and the skin plane. Skin dynamics may result from adhesive peeling, skin wrinkles and maceration, etc. If not properly addressed, skin dynamics may compromise the desired positioning of the microprobe tip during patch use, which could lead to signal instability.

Skin dynamics can also affect the positioning of the microprobe array in several directions in relation to the patch base. Skin dynamics may cause movement in the Y direction or in the direction of any other vectors (Y′).

In order to try and prevent changes in microprobes position due to changes in skin dynamics a suspension-like mechanism is disclosed. A constant residual force (Fr) is applied on the microprobe platform. The constant residual force (Fr) provides some degree of movement in Y′ direction and prevents undesired re-positioning or ejection of the microprobe array vis-à-vis the skin. In the event of partial detachment of the skin from the patch, the force Fr compensates for such displacement and re-positions the microprobe platform 400 to maintain contact between the skin surface and the microprobe skin stopper/microprobe holder.

Since skin dislocation from the patch may occur in multiple different directions, the microprobe platform is designed to allow its rotation in any direction. Maintaining the microprobes at a fixed position in the skin requires a microprobe platform 400 that can adapt in accordance with skin dynamics. In some embodiments, the platform 400 may be a floating platform that may act to reduce the movement of microprobes 208 because of skin dynamics.

In some embodiments a floating mechanism may reduce mechanical coupling between the top shell 102 of the patch 100 and the platform 400. In other words, in some embodiments, the floating mechanism may reduce the mechanical coupling between the internal section and the external section of the patch.

In some embodiments, the floating mechanism may permit the platform 400 to sustain a relative motion between the internal and external sections of the patch without suffering from microprobe dislodgment from the skin. In some embodiments, such relative motion of the platform includes one or more of the following movements: a lateral displacement, a torsional movement, a rotational movement of the internal patch section with respect to the external one. The floating mechanism may be any mechanism that permits the platform 400 to move in one or more of these manners without the microprobes 208 dislodging from the skin. For example, in some embodiments, the floating mechanism is a flexible connector which connects the platform 400 to the top shell 102 of the patch.

In some embodiments, the flexible connector is connected such that a movement of the top shell 102 will not cause a matching-force movement in the platform 400.

In some embodiments, the flexible connector may be configured to supply electrical connectivity for a power supply to the platform and electrical signal transmission to the electronics situated at the inner shell body.

In some embodiments, platform 400 may be connected to the top shell 102 using one flexible connector. In other embodiments, the platform 400 may be connected to the top shell 102 using more than one flexible connector.

The flexible connectors may be spring-like connectors and/or may have a triangular, trapezoid, or elongated rectangular shape. The flexible connectors may be made of metal, rubber, or other polymeric materials. In some embodiments the flexible connector may be attached perpendicular to the platform 400. In other embodiments, the flexible connector may be attached to the platform at an angle. The flexible connectors may be attached to the platform 400 using any suitable connection means, including for example, anchoring units.

In addition, the flexible connector in combination with the skin stopper 214 may be configured to help protect the skin while the patch is being work. In some embodiments, the thickness of the microprobe array substrate 414 may be relatively small, potentially forming a sharp edge at the skin-facing side. Applying a residual force on the platform in order to keep the microprobe array substrate 414 surface in contact with the skin may lead to a skin lesion. Moreover, such lesion may be exacerbated due to skin movement or additional external force applied to the patch shell.

The residual force being continuously applied to the patch platform 400 supplemented with the skin stopper 214 may adjust the microprobe position in the Y direction. However, skin dynamics or external movement of the patch may interfere with the microprobe position in the direction of the Y′ axis. Reducing the effect of such forces on microprobe position may be provided by the floating capabilities of the microprobe skin stopper. In such cases, maximal separation of the skin stopper 214 (connected with microprobe platform) from the patch shell 102 is suggested, in order to enable their independent movement.

In some embodiments, an adhesive layer is applied to the skin-facing part of the skin stopper 214, such that the patch base 104 adhesive is separated from the microprobe skin stopper adhesive. Such an embodiment may provide independent motion of the patch shell 102 with respect to the skin stopper 214 and the platform 400.

In use, there may be two states of the patches of the present disclosure, pre-mounted and as-mounted. The as-mounted state of the patch occurs when the patch is adhered to an individual's skin and the microprobes are inserted into the skin. The pre-mounted state describes the patch configuration prior to microprobe insertion. For example, when the patch is mounted on the skin and is ready for microprobe insertion.

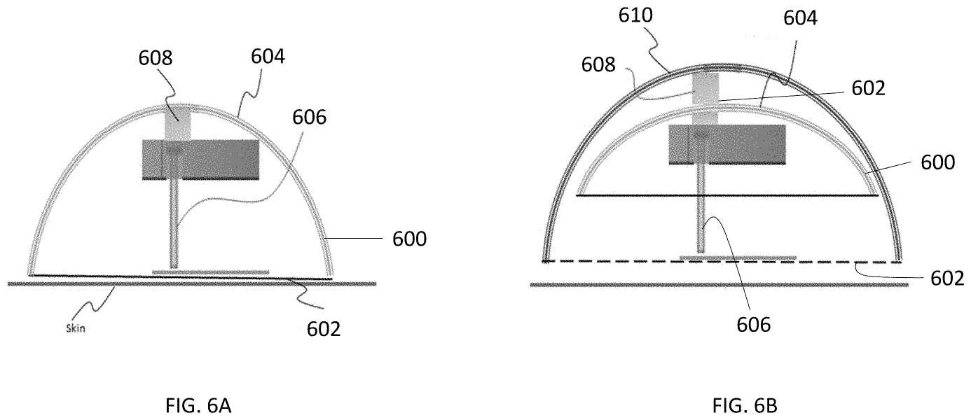

Described herein and depicted in FIGS. 6A and 6B are two exemplary embodiments for a pre-mounted patch configuration.

In the embodiment depicted in FIG. 6A, the pre-mounted patch 600 includes a base 602 and a top shell 604, which may be configured change shape and or position between pre-mounted and mounted states. In this embodiment, the height of the top shell 604 may be greater than the height of the microprobe 606. That is, the height of the patch 600 may be dictated and limited by the length of the microprobe system itself (e.g., less than 1 mm, 2 mm, 3 mm, 4 mm, 5 mm, or 6 mm).

In the embodiment depicted in FIG. 6B, the height of the top shell 604 is slightly greater than the height of the internal components (i.e., the platform 400). In this embodiment, a disposable applicator 610 may surround the top shell 604 and the microprobe 606.

In each embodiment of FIGS. 6A and 6B, an insertion tab 608 aligns the insertion force vector with the axis of the microprobe system. In some embodiments, the insertion tab 608 may be a bulge in the patch top shell 604 or in a disposable applicator 610. In other embodiments, the insertion tab 608 may be a bulge may be on the external and or internal facets of patch top shell 604 or of a disposable applicator 610. The aligned force vector ensures vertical insertion of the microprobes 606 and with minimal force.

In some embodiments, the patch may include a fail-safe mechanism that prevents unintended patch triggering. Such a mechanism may further function to ensure the patch is fastened to the skin before activation. Quality of the initial and ongoing adhesion between the patch base and the skin is an important factor for any skin-mounted patch. In microprobe bearing patches, reaching tight-skin adhesion is critical due to short microprobe length. Even a relatively small sub-mm gap between the patch base and the skin might compromise microprobe insertion into the skin and sensor displacement. In some embodiments, to achieve tight adhesion of the patch to the user skin, intimate contact between the patch base and the skin should be formed before patch activation.

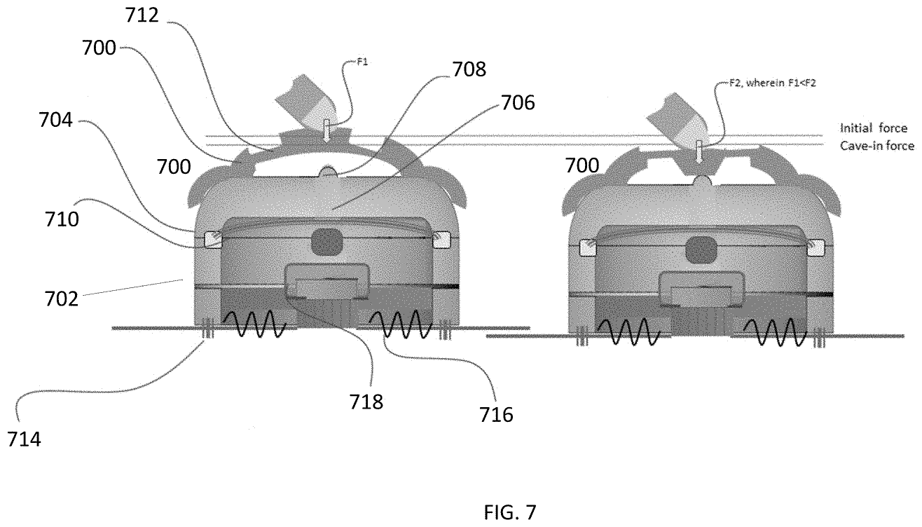

An exemplary embodiment of the fail-safe mechanism that may prevent unintended patch triggered is depicted in FIG. 7. Specifically, FIG. 7 depicts a “cave-in” cover 700 that is placed on top of the patch top shell. The “cave-in” cover 700 may be designed to address two issues: The first such issue, as discussed above, may be to prevent unintended force activation. The second issue may be ensuring that the intended patch activation occurs by applying a predetermined force to the patch prior to force activation.

In some embodiments, the “cave-in” cover 700 may sustain a certain, predetermined force while maintaining its shape. In addition, the “cave-in” cover 700 may be designed to collapse once excessive force, needed to ensure tight adhesion between the patch and the skin, is applied to it. Collapse of the “cave-in” cover 700 may result in activation of the microprobe array insertion into the user's skin.

In some embodiments, the “cave-in” cover 700 may be configured to withstand a force that is greater than 0.1 Kg., 0.2 Kg, 0.3 Kg, 0.5 Kg., 0.75 Kg, 1.0 Kg, 1.25 Kg., 1.5, Kg, 1.75 Kg, 2.0 Kg., 2.5 Kg, or 3.0 Kg.

In addition, in some embodiments, the “cave-in” cover 700 may be configured to “cave-in” when the force is greater than 0.1 Kg., 0.2 Kg, 0.3 Kg, 0.5 Kg., 0.75 Kg, 1.0 Kg, 1.25 Kg., 1.5, Kg, 1.75 Kg, 2.0 Kg., 2.5 Kg, 3.0 Kg.

In some embodiments, the “cave-in” cover 700 is configured for use with a patch 702 having a shell 704 through which an insertion mechanism 706 passes. In some embodiments, the insertion mechanism 706 is coupled to a button 708 that faces the “cave-in” cover 700. In some embodiments, the patch 702 includes a leaf spring 710 that is positioned so as to press against the insertion mechanism 706. In some embodiments, the “cave-in” cover 700 includes fault lines 712 along which the “cave-in” cover 700 is configured to flex and/or fracture when actuated as described above. In some embodiments, the patch 702 includes skin adhesive 714 that is operative to affix the patch 702 to skin. In some embodiments, the patch 702 includes flexible connectors 716 (e.g., springs) that connect the shell 704 to a microprobe platform 718. In some embodiments, when a sufficient force is applied to the “cave-in” cover 700, the “cave-in” cover 700 collapses, resulting in the application of a force via the button 708 and the insertion mechanism 706 and to the leaf spring 710. The leaf spring 710 displaces downward (e.g., toward the skin), thereby causing the microprobe platform 718 to displace downward and position the microprobes in the skin.

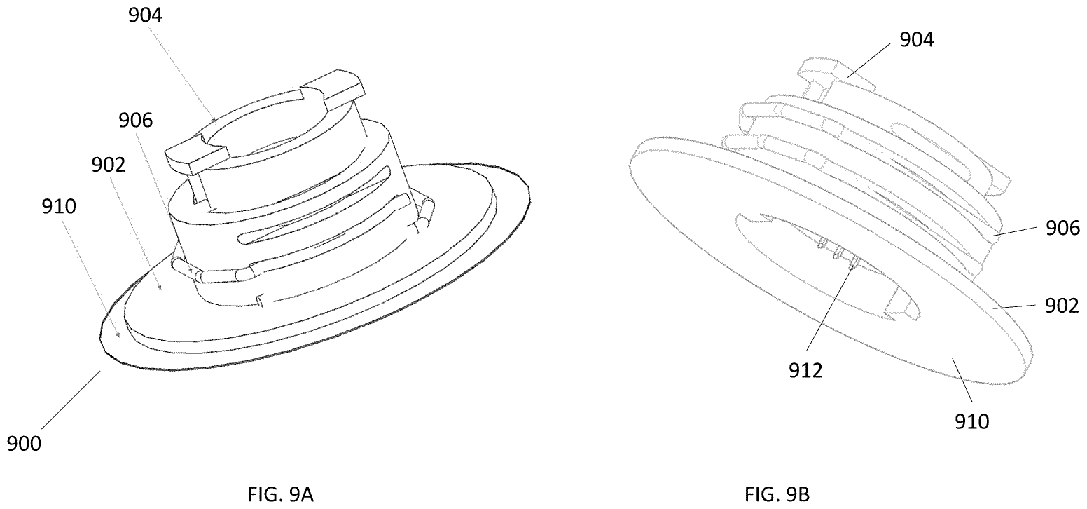

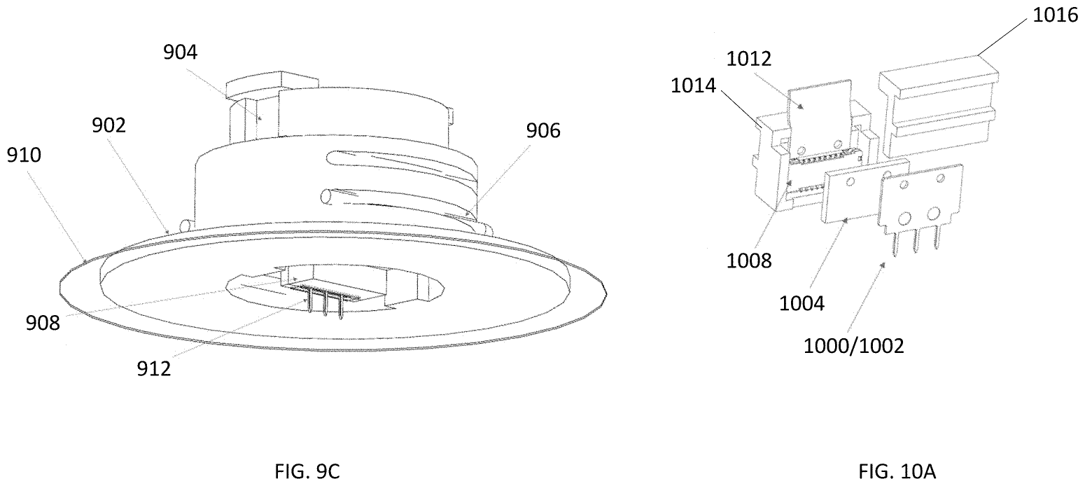

FIGS. 9A-9C show an embodiment of a patch 900. FIG. 9A shows a top perspective view of the patch 900, FIG. 9B shows a bottom perspective view of the patch 900 in a pre-deployment position, and FIG. 9C shows a bottom perspective view of the patch 900 in a deployed position. In some embodiments, the patch 900 includes a generally cylindrical base 902 having a bore with an inner piston-like part 904 slidably positioned therein. In some embodiments, the patch 900 includes a retention spring 906 operatively coupled to a microprobe housing 908 so as to retain the microprobe housing 908 in a pre-deployment position such that microprobes 912 of the microprobe housing 908 are positioned within (e.g., do not protrude from) the base 902, as shown in FIG. 9B. In some embodiments, the retention spring 906 is a U-shaped spring that engages the microprobe housing 908 so as to retain the microprobe housing 908 in its resting position. In some embodiments, an adhesive 910 is positioned at the bottom (e.g., skin-facing) surface of the base 902. In some embodiments, the adhesive 910 is a skin-safe adhesive that is suitable to retain the patch 900 on a person's skin for a period of time.

In some embodiments, to deploy the microprobes 912 of the patch 900, pressure is applied to the inner piston so as to overcome the retention force applied by the retention spring 906. In some embodiments, once the retention force applied by the retention spring 906 is overcome, the inner piston-like part 904 is allowed to travel from its resting position (e.g., as shown in FIG. 9B) to its deployed position (e.g., as shown in FIG. 9C). In some embodiments, once the inner piston-like part 904 has been deployed, the base 902 and the retention spring 906 are removable therefrom and the inner piston-like part 904 is retained on a subject's skin by the adhesive 910.

In some embodiments, the retention spring 906 is configured to allow the microprobe housing 908 to move from the resting position to the deployed position upon the application of a sufficient release force to the piston-like part 904 that is greater than 0.1 Kg., 0.2 Kg, 0.3 Kg, 0.5 Kg., 0.75 Kg, 1.0 Kg, 1.25 Kg., 1.5, Kg, 1.75 Kg, 2.0 Kg., 2.5 Kg, or 3.0 Kg.

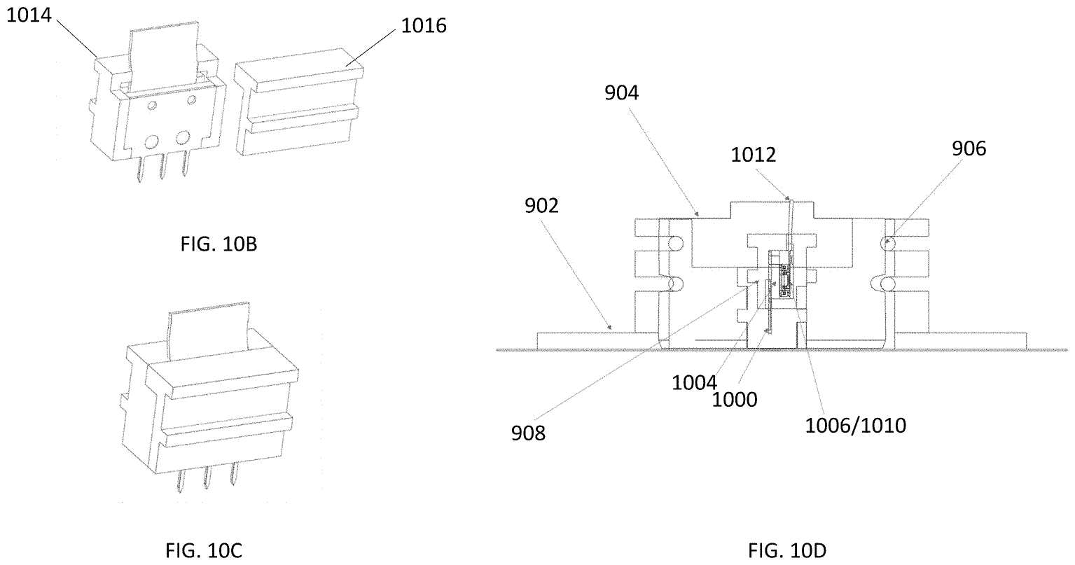

FIGS. 10A-10C show detailed views of the microprobe housing 908. FIG. 10A shows an exploded view of the microprobe housing 908, FIG. 10B shows a partially assembled view of the microprobe housing 908, and FIG. 10C shows an assembled view of the microprobe housing 908. FIG. 10D shows a detailed cross-sectional view of the microprobe housing 908 as positioned within the patch 900. In some embodiments, the microprobe housing 908 includes a microprobe chip 1000 (e.g., a sensing chip) that is attached to a metal microprobe support 1002. In some embodiments, the microprobe support 1002 is configured to support the microprobe chip 1000 in the same manner as described above with reference to the microprobe array support 416. In some embodiments, the microprobe chip 1000 and microprobe support 1002 are attached to a microprobe PCB 1004. In some embodiments, the microprobe chip 1000 is wirebonded to the microprobe PCB 1004. In some embodiments, the microprobe PCB 1004 includes a microprobe PCB connector 1006. In some embodiments, the microprobe PCB connector 1006 is at an opposite end of the microprobe PCB 1004 from the microprobe chip 1000 and faces away from the microprobe chip 1000. In some embodiments, the microprobe PCB connector 1006 is coupled to a second PCB 1008. In some embodiments, the second PCB 1008 has a second PCB connector 1010 that matches and is coupled to the microprobe PCB connector 1006. In some embodiments, the second PCB 1008 is coupled to a flex connector 1012 that is configured to provide a flexible connection between the microprobe chip 1000 (which is movable as described herein) and an external or integral processing and/or recording system. In some embodiments, the microprobe chip 1000, the microprobe support 1002, the microprobe PCB 1004, the second PCB 1008, and the flex connector 1012 are assembled within a housing first part 1014 and a housing second part 1016 to form the microprobe housing 908. In some embodiments, the elements of the microprobe housing 908 are assembled using standard means (e.g., fasteners, snap-together construction, etc.)

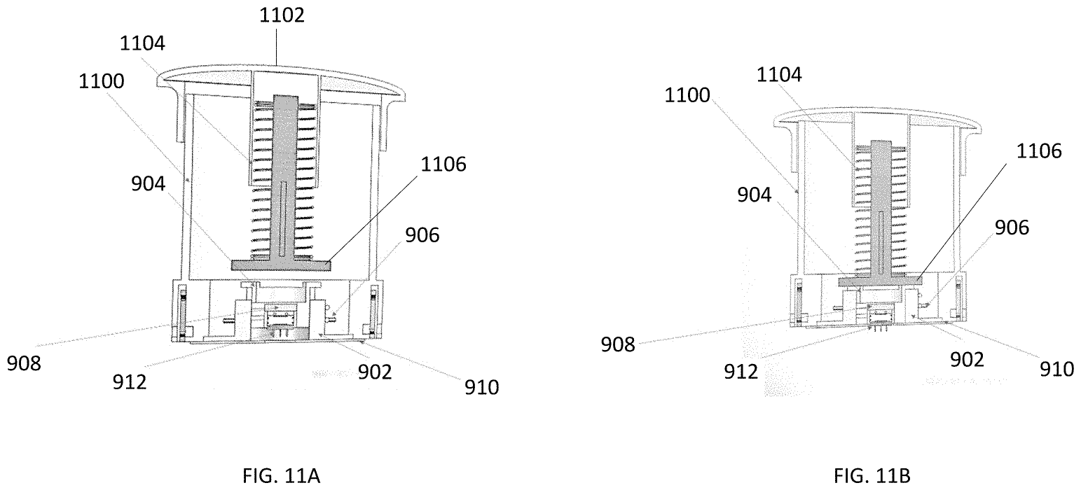

In some embodiments, the patch 900 is used in conjunction with an applicator 1100 that is operable to assist in deploying the microprobes 912 of the patch 900. FIGS. 11A-11B show cross-sectional views of an exemplary embodiment of the applicator 1100 used in conjunction with the patch 900. FIG. 11A shows the applicator 1100 as positioned prior to deployment of the microprobes 912. FIG. 11B shows the applicator 1100 as positioned after deployment of the microprobes 912. In some embodiments, the applicator 1100 includes an actuator 1102 (e.g., a pushbutton) that is operable by a user to deploy the microprobes 912. In some embodiments, the actuator 1102 is operably coupled to a spring 1104, which is configured to drive a plunger 1106 toward the piston-like part 904 to thereby deploy the microprobes 912.

In some embodiments, the spring 1104 is preloaded before it is released. In such embodiments, the patch 900 is first pressed against the skin, and then an individual uses the actuator 1102 to release the spring 1104 and thereby deploy the microprobes 912 into the skin.

In some embodiments, the spring 1104 is not preloaded. In such embodiments, the spring 1104 is connected to the base 902 of the patch 900 and is pressed together with the base 902 against the skin to insert the microprobes 912 into the skin.

It is appreciated that certain features of the disclosure, which are, for clarity, described in the context of separate embodiments, may also be provided in a single embodiment. Conversely, various features of the disclosure, which are, for brevity, described in the context of a single embodiment, may also be provided separately or in any suitable sub combination or as suitable in any other described embodiment of the disclosure. Certain features described in the context of various embodiments are not considered essential features of these embodiments, unless the embodiment is inoperative without those elements.

What is claimed is:

1. A wearable sensor patch, comprising:
a generally cylindrical base having:
a bore, and
a skin contact surface having an adhesive thereon;
a piston-like part positioned within the bore;
at least one microprobe positioned on the piston-like part; and
a retention spring;
wherein the piston-like part is movable within the bore of the base between (1) a first position in which the at least one microprobe is positioned within the bore, and (2) a second position in which the at least one microprobe protrudes past the skin contact surface, and
wherein the retention spring and the piston-like part are configured to cooperate such that the retention spring retains the piston-like part in either the first position or the second position.

2. The wearable sensor patch of claim 1, wherein the retention spring is configured to allow the piston-like part to move from the first position to the second position upon application of a sufficient release force to the piston-like part.

3. The wearable sensor patch of claim 2, wherein the sufficient release force is at least 0.1 kilogram.

4. The wearable sensor patch, of claim 1, wherein the at least one microprobe is positioned within a microprobe housing.

5. The wearable sensor patch of claim 4, wherein the microprobe housing comprises a flex connector configured to connect the at least one microprobe to an external processing system.

6. The wearable sensor patch of claim 4, wherein the microprobe housing comprises a metal support supporting the at least one microprobe.

7. The wearable sensor patch of claim 4, wherein the microprobe housing includes a housing first part and a housing second part encasing at least part of the at least one microprobe.

8. (canceled)

9. A sensor system, comprising:
the wearable sensor patch of claim 1; and
an applicator.

10. The sensor system of claim 9, wherein the applicator comprises:
an actuator configured to be actuated by a user;
a plunger configured to contact the piston-like part of the wearable sensor patch when the applicator is assembled to the wearable sensor patch; and
a spring configured to apply a force to the plunger when the actuator is actuated.

11. The sensor system of claim 10, wherein the spring is pre-loaded.

12. The sensor system of claim 10, wherein the spring is configured to be loaded upon application of the wearable sensor patch to skin of a user.

13. The wearable sensor patch of claim 1, wherein the at least one microprobe comprises a plurality of microprobes.

14. The wearable sensor patch of claim 13, wherein the plurality of microprobes are staggered so as to include a lead microprobe that contacts the skin before an additional microprobe.

15. (canceled)

16. (canceled)

17. A wearable sensor patch, comprising:
a casing;
a movable microprobe platform movably positioned within the casing, wherein the movable microprobe platform includes a microprobe system; and
a multiple-spring microprobe array insertion mechanism, comprising:
a plurality of springs, and
a latch operable to activate the multiple-spring microprobe array insertion mechanism,
wherein, when the latch is operated so as to activate the multiple-spring microprobe array insertion mechanism, the plurality of springs acts on the movable microprobe platform with a combined force of the plurality of springs so as to deploy the movable microprobe platform.

18. The wearable sensor patch of claim 17, wherein the multi-spring microprobe array insertion mechanism further comprises a spring motion limiter,
wherein the multiple-spring microprobe array insertion mechanism includes a first spring and a second spring, and
wherein the spring motion limiter is configured to limit a travel of the first spring such that the first spring stops applying force to the movable microprobe platform when the first spring contacts the spring motion limiter while the second spring continues to be in contact with the movable microprobe platform and to apply a force of the second spring to the movable microprobe platform.

19. The wearable sensor patch of claim 18, wherein the second spring is configured to apply an ejection-counterforce sufficient to resists force that may move microprobes of the microprobe system from a skin position or eject the microprobes from skin.

20. The wearable sensor patch of claim 19, wherein the first spring is configured to apply a force that is more than two times the ejection-counterforce.

21. The wearable sensor patch of claim 17, wherein a combined force applied by the plurality of springs is at least 50 grams.

22. The wearable sensor patch of claim 17, wherein the plurality of springs includes a plurality of torsion springs.

23. (canceled)

24. The wearable sensor patch of claim 17, further comprising a safety mechanism configured to prevent unintended release of the multiple-spring microprobe array insertion mechanism.
